(19) United States
(12) Patent Application Publication
(10) Pub. No.: US 2010/0211019 A1
(43) Pub. Date: Aug. 19, 2010

(54) CLOSURE DEVICE FOR LINES FOR ADMINISTERING MEDICAL OR PHARMACEUTICAL FLUIDS FROM CONTAINERS OR THE LIKE

(76) Inventor: Francesco Greco, Cantu (IT)

Correspondence Address: Modiano & Associati, Via Meravigil 16, Milano, 20123, IT

(21) Appl. No.: 12/452,664

(22) PCT Filed: Jan. 4, 2008
(86) PCT No.: PCT/IT2008/000010

§ 371 Date: Jan. 14, 2010

(30) Foreign Application Priority Data
Jul. 19, 2007 (IT) MO2007A000240

(51) Int. Cl. A61M 39/22; B67C 3/20

(52) U.S. Cl. 604/246; 141/21

(57) ABSTRACT

A device for closing lines for administering medical or pharmaceutical fluids from containers and the like, comprising: a first tubular body and a second tubular body, which are mutually associated and able to move axially by sliding and/or rotating with respect to each other; a valve element for adjusting the flow of the fluid formed inside the first and second tubular bodies, the relative movement of the first and second tubular bodies being adapted to move the valve element from a closed configuration for blocking the flow of the fluid to an open configuration for feeding the fluid; elements for limiting the relative rotation between the first and second tubular bodies which are monolithically connected to at least one of the first and second tubular bodies.

The present invention relates to a closure device for lines for administering medical or pharmaceutical fluids from containers and the like, particularly syringes, bags, catheters, tubes and the like.

### BACKGROUND OF THE INVENTION

In particular, the device according to the invention is used in medical fields in which the medical or pharmaceutical fluids have a high degree of toxicity or irritancy, for example chemotherapy fluids, or other drugs used to treat oncologic disorders.

The individual doses of such drugs to be administered to the patient are generally prepared by specialized medical staff of the medical facility. In particular, the drugs are packaged in containers, for example bags or bottles, from which specialized staff, with the aid of conventional syringes, draws the individual doses to be administered to the patient by means of suitable administration devices, such as for example phleboclyses, catheters or the like.

It is necessary to avoid accidental leaks or losses of the medical or pharmaceutical fluids during the steps for their preparation, transport and administration, since such leaks might cause intoxications or irritations to anyone coming into contact with them.

Closure devices for lines for administering medical or pharmaceutical fluids from containers, particularly syringes, bags, catheters, tubes and the like are known which have two tubular bodies which are mutually associated at a respective end, are mutually substantially coaxial and can move axially so as to slide and/or rotate with respect to each other.

The tubular bodies are open at their respective opposite ends, which have for example couplings of the Luer-Lok type which can be associated respectively with the mouth of a container or with a line for administering a fluid and to the mouth of a reservoir for supplying the fluid to the container, or to the administration line, or to the intake port of a fluid user device.

Inside the tubular bodies there is a valve element for adjusting the flow of the fluid. In particular, the relative movement of the tubular bodies is adapted to move the valve element from a closed configuration for blocking the flow of the fluid to an open configuration for the passage of the fluid.

Finally, the known type of closure device comprises means for temporary locking in an open and/or closed configuration, which are interposed between said first and second tubular elements and can be actuated by an operator by means of a relative rotation of the tubular elements.

In particular, the locking means are provided by way of a pair of pins, which are associated externally with one of the two tubular bodies and are adapted to interfere with two wings, provided with suitable receptacles, which are associated externally with the other of the two tubular bodies.

In particular, the locking means act by interrupting temporarily the relative axial translational stroke of the two bodies.

These known types of closure device are in any case not free from drawbacks, including the fact that the wings for locking temporarily the relative axial translational stroke of the two bodies have, in some operating conditions, limited mechanical resistance to wear and break and/or lose efficiency in the short term.

Further, it has been observed in known types of device that the pins associated with one of the tubular bodies, if subjected to vigorous rotation by the specialized user, can bend, for example in a radial direction, the wings for their retention, thus thwarting their locking action.

### SUMMARY OF THE INVENTION

The aim of the present invention is to eliminate the drawbacks cited above of the background art, by providing a closure device for lines for administering medical or pharmaceutical fluids from containers, particularly syringes, bags, catheters, tubes and the like, which allows to increase the mechanical resistance and efficiency of the temporary locking means.

Within this aim, an object of the invention is to reduce the mutual rotation stroke between the tubular bodies, improving the mating efficiency of the pins with the respective receptacles.

Another object of the invention is to protect the mutually moving elements, in particular the two tubular bodies, against agents which are external to said closure device.

Another object of the present invention is to provide a closure device which is simple, relatively easy to provide in practice, safe in use, effective in operation, and has a relatively low cost.

This aim and these and other objects, which will become better apparent hereinafter, are achieved by the present device for closing lines for administering medical or pharmaceutical fluids from containers and the like, which comprises:
a first tubular body and a second tubular body, which are mutually associated at a respective end and are open at the respective opposite ends, said first and second tubular bodies being mutually substantially coaxial and able to move axially by sliding and/or rotating with respect to each other, said first and second tubular bodies having respective free ends associable respectively with the mouth of a container or with a line for administering a fluid and with the mouth of a reservoir for supplying said fluid to said container or to said administration line or to the inlet of a fluid user device,
a valve element for adjusting the flow of said fluid formed inside said first and second tubular bodies, the relative movement of said first and second tubular bodies being adapted to move said valve element from a closed configuration for blocking the flow of said fluid to an open configuration for feeding said fluid from said reservoir to said container, or to said administration line, or from said container, or from said administration line, to said user device, and vice versa,
means for temporary locking in an open and/or closed configuration, which are interposed between said first and second tubular bodies and can be operated by an operator by means of a relative rotation of said tubular bodies,

characterized in that it comprises means for limiting the relative rotation between said first and second tubular bodies, said limiting means being monolithically connected to at least one of said first and second tubular bodies.

### BRIEF DESCRIPTION OF THE DRAWINGS

Further characteristics and advantages of the present invention will become better apparent from the following detailed description of a preferred but not exclusive embodiment of a device for closing lines for administering medical or pharmaceutical fluids from containers, particularly syringes, bags, catheters, tubes and the like, illustrated by way of non-limiting example in the accompanying drawings, wherein:

FIG. 1 is an exploded perspective view of a closure device according to the invention;

FIG. 2 is an exploded side view of the closure device;

FIG. 3 is a perspective view of the assembled device in the closed configuration, with the locking means engaged;

FIG. 4 is a perspective view of the assembled device in the open configuration, with the locking means engaged;

FIG. 5 is a perspective view, similar to FIG. 4, in a configuration for the disengagement of the locking means;

FIG. 6 is an exploded perspective view of an alternative embodiment of the device according to the invention, in the open configuration;

FIG. 7 is a view of a detail of the device of FIG. 6 in the closed configuration;

FIG. 8 is a view of a detail of the device of FIG. 6 during the step for passing from the open configuration to the closed configuration or vice versa;

FIG. 9 is a perspective view of the device of FIG. 6 in the assembled condition.

### DESCRIPTION OF THE PREFERRED EMBODIMENTS

With reference to the figures, the reference numeral 1 generally designates a device for closing lines for administering medical or pharmaceutical fluids from containers or the like, such as for example bottles, phleboclyses, catheters and others.

The device 1 comprises a first tubular body 2 and a second tubular body 3, which can be mutually associated at a respective end 2a and 3a and are open at least at respective opposite ends 2b and 3b.

The first tubular body 2 and the second tubular body 3 are mutually substantially coaxial and can move axially by sliding along a direction D1 and/or by rotating with respect to each other along a direction R1.

In a preferred embodiment, shown in the figures, the first and second tubular bodies 2 and 3 can move with respect to each other both by rotating and by sliding.

The first and second tubular bodies 2 and 3, in particular, have the respective opposite ends 2b and 3b which are free and can be associated, for example by means of devices of the male and/or female Luer-Lok type or others, respectively with the mouth of a container or with a line for administering a fluid or with the mouth of a reservoir for supplying said fluid to the container or with the administration line or the inlet port of a fluid user device, which are not shown in the figures since they are of a type which is known to the person skilled in the art.

Moreover, the device 1 comprises a valve element for adjusting the flow of the fluid, also not shown in the figures since it is of a known type, which is formed inside the first and/or second tubular bodies 2 and 3.

In particular, the relative movement of the first and second tubular bodies 2 and 3 is adapted to move the valve element from a closed configuration for blocking the flow of the fluid to an open configuration for the inflow of said fluid from the reservoir to the container or to the administration line, or from the container or from the administration line to the user device, and vice versa.

By way of non-limiting example, the valve element can be of the type of a flow control element S or the like, which for example has a stem which is accommodated within the first and/or second tubular body 2 and 3 and is substantially coaxial to them, one end being associated with at least one of the first and second tubular bodies while the other end has a head which can be inserted hermetically in a cavity C of the other of the first and second tubular bodies 2 and 3. However, alternative and equivalent embodiments of the valve element and/or of the flow control element are not excluded.

The free end 2a of the first tubular body 2 is contoured so as to form a plurality of radial ribs blended with a circumferential ridge, in the lateral edge of which a sealing ring T is accommodated, said ring being adapted to interfere with the internal wall of the second tubular body 3 in order to avoid the escape of fluid and constituting part of the stop means.

In particular, for example, said stop means have a first annular groove and a second annular groove, not visible in the figures, which are formed on the internal wall of the second tubular body 3 and are spaced by an extent which corresponds to the relative stroke between the first and second tubular bodies 2 and 3 in order to pass from the open configuration to the closed configuration and vice versa. In said extreme configurations, the sealing ring T engages alternately one of said annular grooves, limiting the relative sliding between the first and second tubular bodies.

A plurality of recesses R spaced out with the radial ribs is formed at the end 2a of the first tubular body 2.

During withdrawal/dispensing, with the device 1 in the open configuration in which the flow control element disengages the cavity C, the fluid passes through the recesses R and flows inside the first tubular body 2.

The device 1 further comprises means for temporary locking in the open and/or closed configuration, which are interposed between the first and second tubular bodies 2 and 3 and can be actuated by an operator by means of a relative rotation of said first and second tubular bodies.

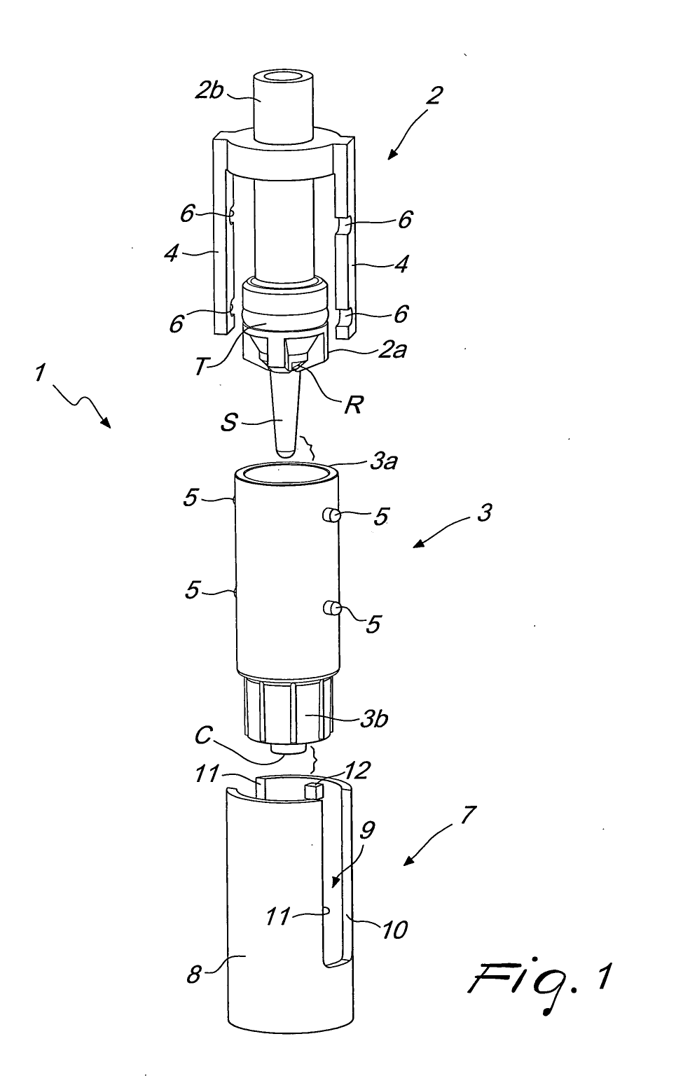

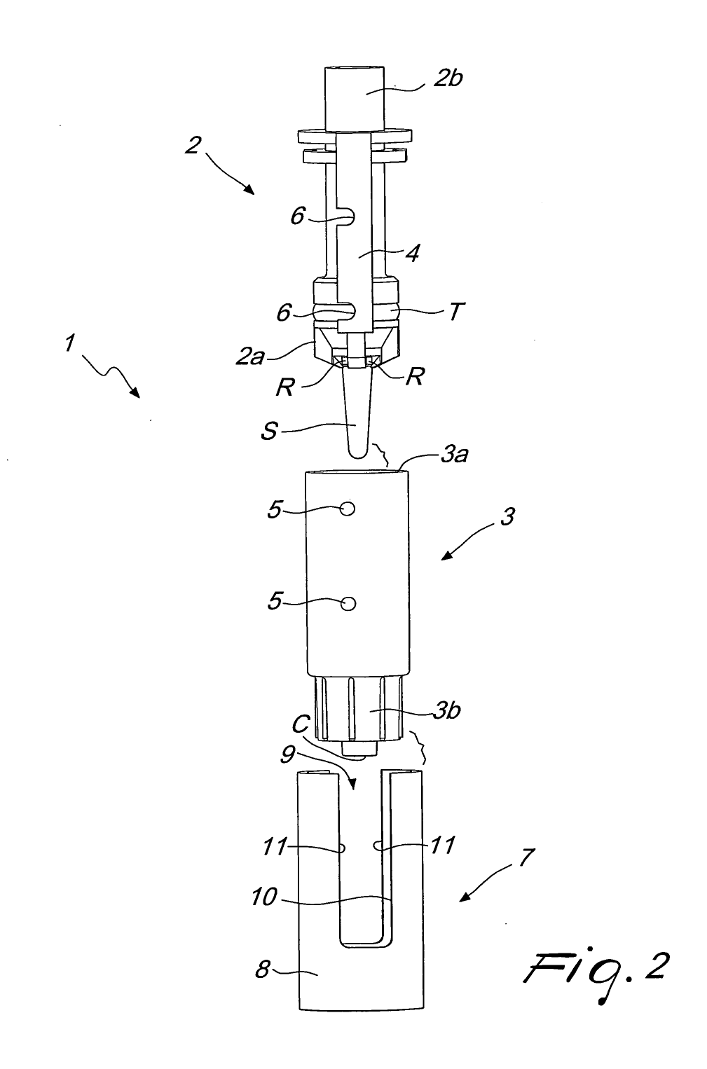

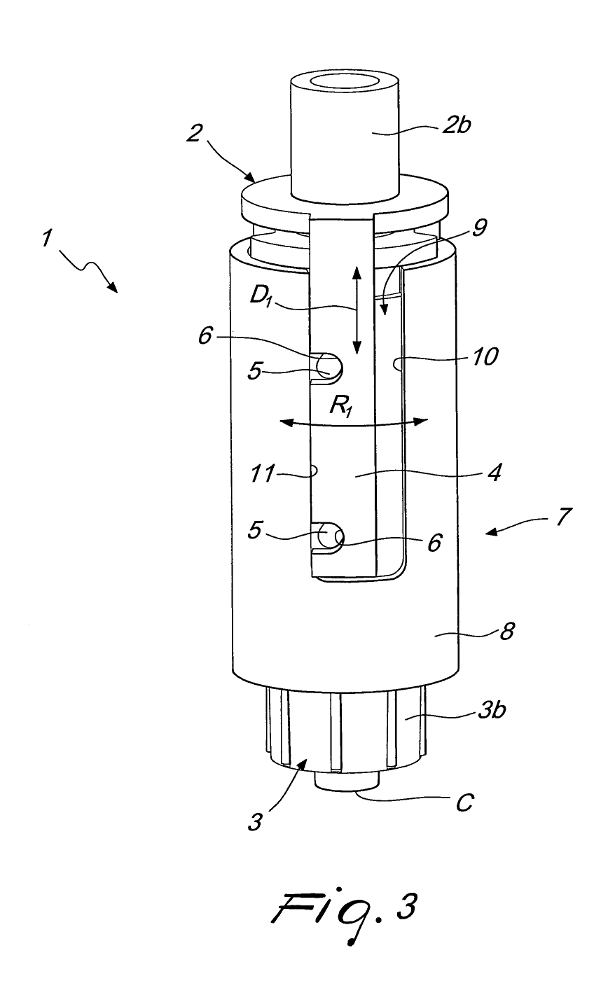

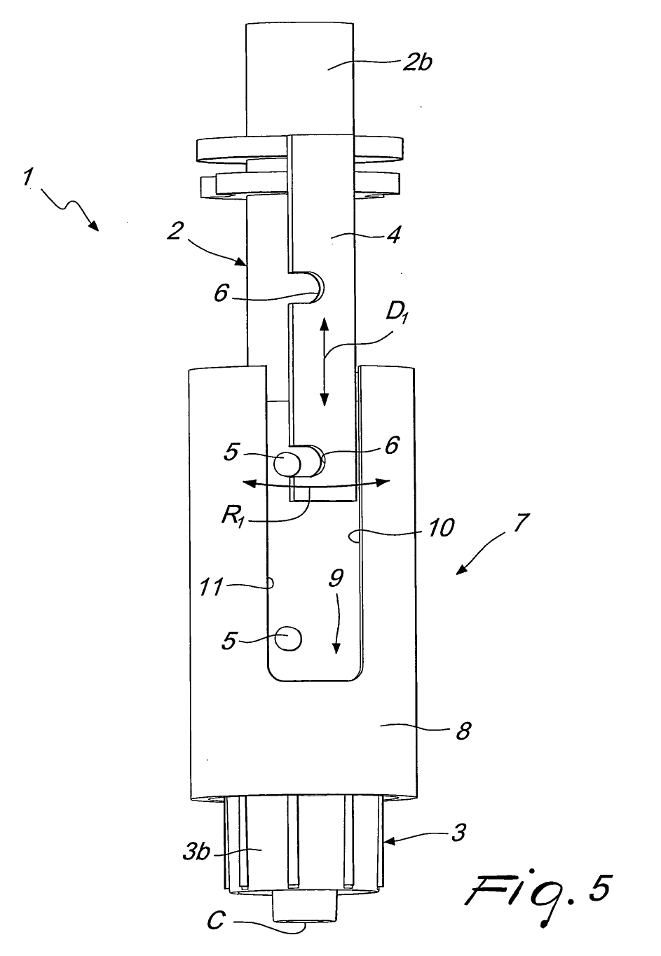

In a first embodiment, shown in FIGS. 1 to 5, advantageously the temporary locking means comprise at least one wing 4, which protrudes from the first tubular body 2 and is substantially parallel to the axis of said first tubular body.

The wing 4 can be superimposed on the outer surface of the second tubular body 3.

The temporary locking means comprise at least one pin 5, which is associated with at least one of the first tubular body 2 and the second tubular body 3 and is adapted to engage in at least one receptacle 6 which is associated with the other of said first and second tubular bodies.

The locking means comprise at least one pair of receptacles 6 for temporarily retaining said pin respectively in the open configuration and in the closed configuration.

The pin 5 protrudes from said outer surface of the second tubular body 3 for insertion in the at least one receptacle 6 of the pin 5 provided on the wing 4.

In particular, the temporary locking means comprise at least one pair of pins 5, which are aligned along the second tubular body 3 and are spaced by an extent which is equal to the relative sliding stroke between the first and second tubular bodies 2 and 3.

The wing 4 is provided with a pair of receptacles 6 adapted to accommodate at least one of the pins 5.

However, equivalent alternative embodiments of the temporary locking means are not excluded.

In particular, in the closed configuration, with the locking means engaged, both pins 5 are accommodated within both receptacles 6, while in the open configuration and with the locking means engaged, a single pin 5, in particular the distal one with respect to the opposite end 3b of the second body 3, is accommodated within a single receptacle 6, in particular the distal receptacle with respect to the opposite end 2b of the first tubular body 2; in the open configuration, the opposite ends 2b and 3b respectively of the first and second tubular bodies 2 and 3 are mutually spaced as a consequence of the relative sliding movement of said tubular bodies.

Particularly, in order to achieve the above-mentioned aim and objects, the device 1 comprises means 7 for limiting the relative rotation between the first and second tubular bodies 2 and 3.

The limiting means 7 are rigidly connected to at least one of the first and second tubular bodies 2 and 3.

In particular, in the first embodiment shown in FIGS. 1-5, the limiting means 7 are fixed externally to at least one of the first and second tubular bodies 2 and 3.

The limiting means 7 advantageously comprise at least one third tubular body 8, which is rigidly connected, externally and coaxially, to the second tubular body 3. However, different embodiments of the third tubular body 8 are not excluded, and it can be for example rigidly connected to the first tubular body 2.

The third tubular body 8 is provided with at least one notch 9 which is arranged at the wing 4.

In particular, the third tubular body 8 has a substantially cylindrical hollow shape and the notch 9 has a longitudinal axis which is substantially parallel to the axis of said third tubular body.

Advantageously, the notch 9 comprises at least one first lateral wall 10 for the abutment of the wing 4 in a configuration, referenced as locking means disengagement configuration, in which the pins 5 are both uncoupled from the respective receptacles 6.

In particular, the first wall 10 substantially faces the wing 4 on the opposite side with respect to the side provided with the receptacles 6.

Further, the notch 9 comprises a second wall 11, which instead faces the receptacles 6.

In particular, the second wall 11 is separated by a specific circumferential portion from the wing 4 in the locking means disengagement configuration.

In particular, the first and second walls 10 and 11 are adapted to interfere with the wing 4 in order to determine the relative rotation stroke limit, in the two directions of rotation, between the first and second tubular bodies 2 and 3.

The notch 9 has a circumferential width which is at least greater than the width of the wing 4 and in particular the difference between the width of the notch 9 and the width of the wing 4 is substantially equal to the mutual rotation stroke between the first and second tubular bodies 2 and 3.

In the particular embodiment shown in FIGS. 1 to 5, the relative rotation stroke between the first and second tubular bodies 2 and 3 is further at least substantially equal to the depth of each receptacle 6; in particular, the pins 5 remain in contact with the wing 4 during the relative sliding motion between said first and second tubular bodies.

However, different embodiments, in which for example the third tubular body 8 is rigidly connected to the first tubular body 2 and in which the second wall 11 is partially superimposed on the wing 4, in order to retain it radially, are not excluded.

In this particular embodiment, the pins 5 have, for example, such a length that they can interfere with the first wall 10 and with the receptacle 6 depending on the direction of mutual rotation imparted to the first and second tubular bodies 2 and 3.

In a preferred but not exclusive embodiment, the third tubular body 8 is rigidly connected to the second tubular body 3 by interposing an interlocking coupling between the internal surface of the third tubular body 8 and the external surface of the second tubular body 3.

In particular, the interlocking coupling comprises a pair of teeth, not shown in the figures, which are made for example of elastically flexible material, and are associated with the internal surface of the third tubular body 8 and adapted to be accommodated in a respective pair of grooves formed on the outer surface of the second tubular body 3, for example at the end 3b.

Advantageously, the third tubular body 8 is fixed to the second tubular body 3 by interposing at least one adhesive element, not shown in the figures, which is interposed for example between the third tubular body 8 and said second tubular body at the region affected by the coupling.

The adhesive element can be for example of the type of hot-melt glue; however, different equivalent solutions for fixing the third tubular body 8 to the first tubular body 2 are not excluded, for example ones in which the third tubular body 8 is provided monolithically with said first tubular body.

Advantageously, in order to determine the uniform distribution of the torsional forces on the first and second tubular bodies 2 and 3, the first tubular body 2 comprises at least one pair of wings 4, each of which is provided with a pair of receptacles 6 and arranged for example on the opposite side with respect to the axis of said first tubular body; the second tubular body 3 comprises at least two pairs of pins 5, which can face the respective receptacles 6; the third tubular body 8 comprises at least one pair of notches 9 which, as described earlier, are arranged at the two wings 4.

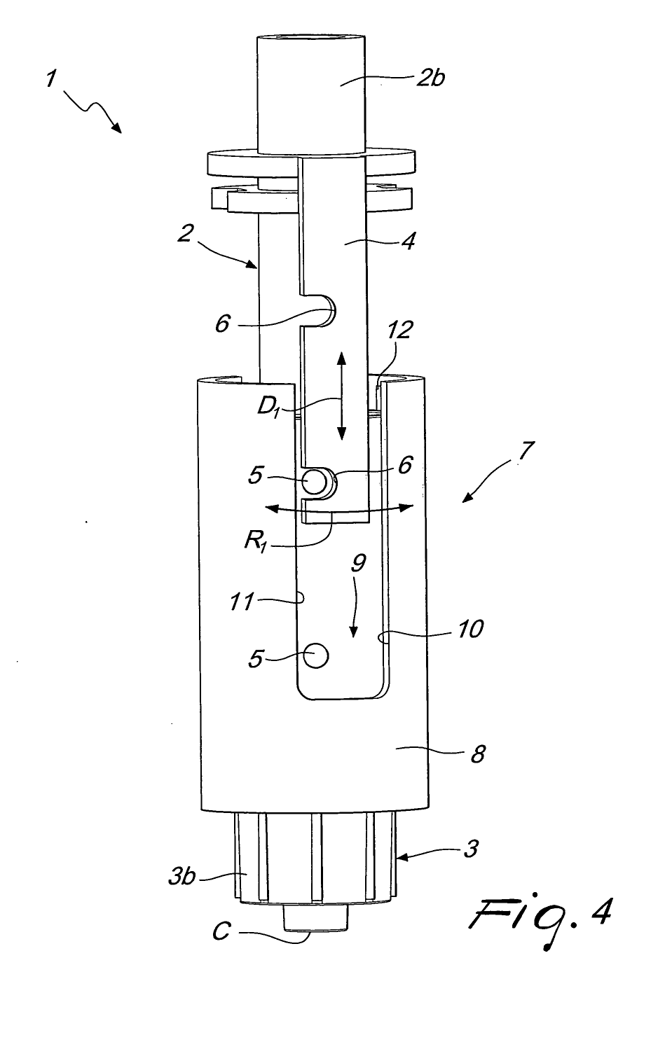

In order to facilitate the operations for mutually fixing the second and third tubular bodies 3 and 8, the latter can have an element for limiting the relative stroke between them, which is fixed for example to the internal surface of the third tubular body 8. The stroke limiting element is, as can be seen in particular in FIGS. 1 and 4, of the type of a block 12, which is adapted to interfere with the end 3a of the second tubular body 3 and determine the relative position between said second and third tubular bodies in cooperation with the interlocking coupling, with the device 1 assembled, for the mutual connection operations described earlier.

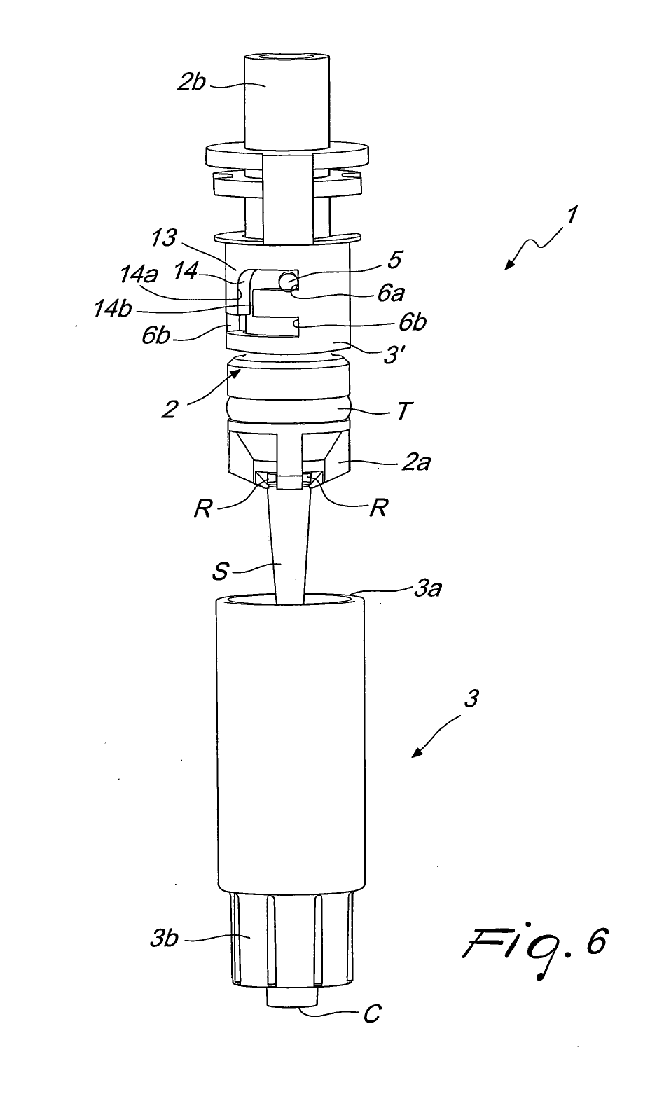

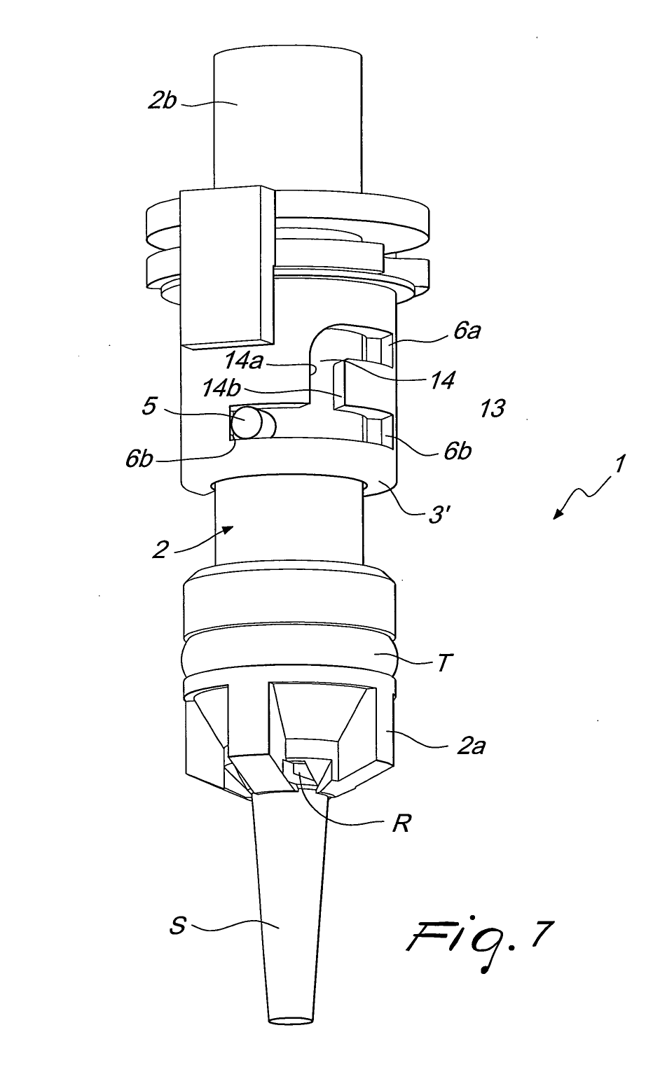

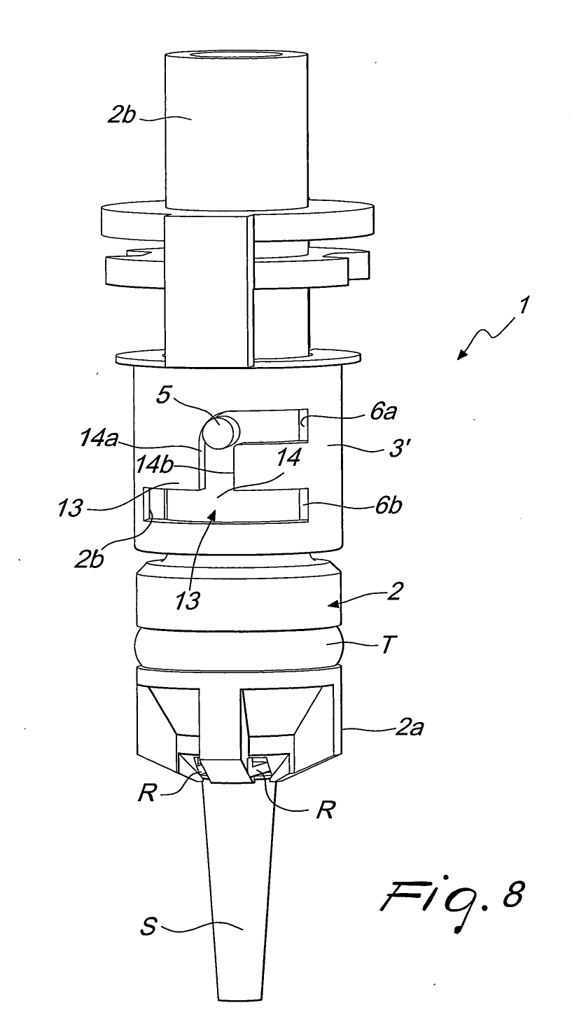

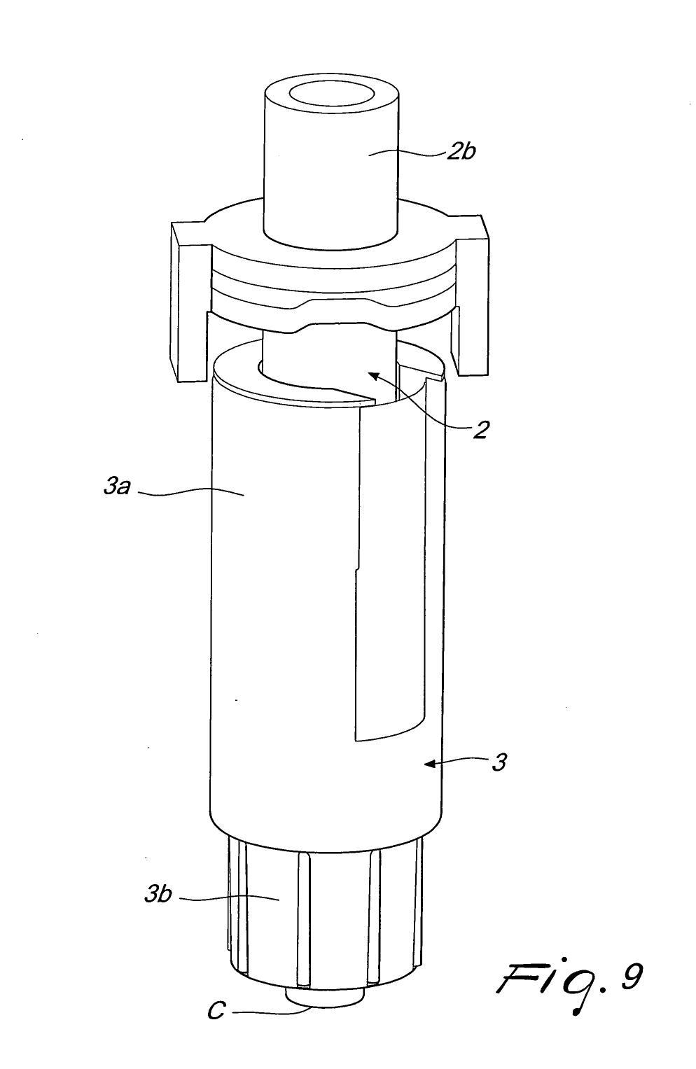

In a second embodiment, shown in FIGS. 6 to 9 and studied in order to reduce the number of components that form the device 1, the locking means are formed monolithically with the first tubular body 2 and with the second tubular body 3.

In this case also, the temporary locking means comprise at least one pin 5 which is associated with at least one of the first and second tubular bodies 2 and 3 and is adapted to engage in at least one receptacle 6 which is associated with the other of said first and second tubular bodies and further comprise at least one pair of receptacles 6 for temporarily retaining said pin respectively in the open configuration and in the closed configuration.

In particular, the second tubular body 3 comprises a tubular portion 3′, which is coupled inside the second tubular body 3 and is rigidly associable therewith, for example by means of interlocking couplings or other technically equivalent solutions.

In turn, the tubular portion 3′ can be fitted onto the first tubular body 2 and can slide axially with a coupling of the cup-like type with play on said first tubular body.

However, different embodiments, in which for example the second tubular body 3 and the tubular portion 3′ are provided monolithically, are not excluded.

In particular, the locking means comprise a single pin 5 which is rigidly associated with the first tubular body 2 and at least one pair of receptacles, respectively 6a and 6b, for accommodating the single pin 5, respectively in the open configuration and in the closed configuration and formed directly on a central region of the second tubular body 3 and in particular of the tubular portion 3′ associated therewith.

The pin 5 is rigidly associated with the outer surface of the second tubular body 3 at an intermediate region thereof and is adapted to be accommodated selectively in one of the receptacles 6a and 6b respectively in the open configuration and in the closed configuration.

However, equivalent embodiments of the locking means are not excluded in which the pin 5 is rigidly associated with the internal surface of the second tubular body 3 and the receptacles are provided in the first tubular body 2.

Advantageously, the locking means comprise two receptacles 6b for the pin 5 in the closed configuration; the receptacles 6b in particular are mutually opposite and are connected for locking the pin 5 in two different positions depending on the direction of mutual rotation imparted between the first and second tubular bodies 2 and 3 in the closed configuration.

The means for limiting the mutual rotation between the first and second tubular bodies 2 and 3 advantageously comprise a guide 13, which is associated with the second tubular body 3 and in particular with the tubular portion 3′, along which there are the receptacles 6a and 6b and in which the pin 5 is inserted so that it can slide.

The guide 13 is provided with at least one intermediate portion 14 for connection between at least one receptacle 6b and the receptacle 6a of the pin 5 for the passage of said pin from at least one of the receptacles 6b in the closed configuration to the receptacle 6a in the open configuration and vice versa, the receptacles 6a and 6b being formed at the ends of the guide 13.

In a preferred embodiment, shown in FIGS. 6 to 9, the connecting portion 14 has a substantially rectilinear longitudinal extension, which is parallel to the axis of the second tubular body 3; however, different embodiments are not excluded in which the connecting portion 14 has a substantially curved longitudinal extension, for example a helical or other shape.

In particular, the pin 5 engages with play the guide 13 during transition from the closed configuration to the open configuration and vice versa.

In the particular embodiment shown in FIGS. 6-9, the total relative rotation stroke between the first and second tubular bodies 2 and 3 is substantially equal to twice the depth of each receptacle 6b and 6a; in particular, in the closed configuration the mutual rotation between the first tubular body 2 and the second tubular body 3 is substantially equal to the distance between the two receptacles 6b and therefore substantially equal to twice the depth of each receptacle 6b.

The pin 5 instead remains in contact with the side walls 14a and 14b of the connecting portion 14 during the movement for mutual sliding between said first and second tubular bodies from the open configuration to the closed configuration and vice versa; accordingly, the mutual rotation between the first and second tubular bodies 2 and 3 is substantially nil, or at most equal to the play provided for the sliding of the pin 5 in the connecting portion 14 of the guide 13 during the transfer of said pin between one of the receptacles 6b and the receptacle 6a and vice versa.

In the open configuration, the stroke for mutual rotation between the first and second tubular bodies 2 and 3 is instead substantially equal to the distance between the receptacle 6a and the side wall 14a of the connecting portion 14 which lies opposite the receptacle 6a.

The operation of the present invention is as follows.

As regards the first embodiment of the device 1, in particular, in the closed configuration, in which the valve element blocks the flow of the fluid, and in the first condition for use of the device 1, the locking means are engaged and therefore both pairs of pins 5 are accommodated in the respective receptacles 6.

In particular, the first wall 10 of the third tubular body 8 is separated in a circumferential direction from the wing 4 by an extent which is equal to the stroke of relative rotation allowed between the first and second tubular bodies 2 and 3.

In the open configuration, in which the valve element is open for the flow of the fluid from one of the opposite ends 2b and 3b to the other, of the first and second tubular bodies 2 and 3 respectively, only the pins 5 which are distal with respect to the opposite end 3b of the second tubular body 3 are accommodated in the respective receptacles 6 which are distal with respect to the opposite end 2b of the first tubular body 2, and therefore in this case also the locking means are engaged.

In this configuration, the first wall 10 of the third tubular body 8 is separated in a circumferential direction from the wing 4 by a portion which is equal to the relative rotation stroke allowed between the first and second tubular bodies 2 and 3.

In order to pass from the closed configuration to the open configuration and vice versa it is necessary to activate the mutual sliding of the first and second tubular bodies 2 and 3.

To do this, it is therefore necessary to pass to the disengagement configuration of the locking means; the first and second tubular bodies 2 and 3 are actuated with a mutual rotation along the direction R1 for the mutual spacing of the pins 5 from the receptacles 6 and the simultaneous movement of the wing 5 toward the first wall 10. In this disengagement configuration of the locking means it is possible to activate only the mutual sliding of the first and second tubular bodies 2 and 3.

The mutual rotation stroke between the first and second tubular bodies 2 and 3 is adjusted in particular by the difference between the width of the notch 9 and the width of the wing 4, which in the particular embodiment shown in the figures is substantially equal to the depth of the receptacles 6.

As regards instead the second embodiment of the device 1, operation is entirely similar to what has been shown for the first embodiment.

In particular, the pin 5, during transition from the open configuration to the closed configuration and vice versa, is guided translationally by the guide 13.

Once in the closed or open configuration, by means of a mutual rotation between the first and second tubular bodies 2 and 3, the pin 5 engages respectively one of the receptacles 6b or the receptacle 6a to temporarily retain it in said chosen configuration.

In practice it has been found that the described invention achieves the proposed aim and objects and in particular the fact is stressed that the closure device for lines for administering medical or pharmaceutical fluids from containers, particularly syringes, bags, catheters, tubes and the like, allows to increase the mechanical strength and efficiency of the temporary locking means.

Moreover, the closure device according to the invention allows to reduce the mutual rotation stroke between the tubular bodies, improving the efficiency of the coupling of the pins with the respective receptacles.

Finally, the invention as conceived allows to achieve a protection against agents which are external to said closure device for the mutually moving elements, in particular the two tubular bodies.

The invention thus conceived is susceptible of numerous modifications and variations, all of which are within the scope of the appended claims.

All the details may further be replaced with other technically equivalent elements.

In practice, the materials used, as well as the contingent shapes and dimensions, may be any according to requirements without thereby abandoning the scope of the protection of the appended claims.

The disclosures in Italian Patent Application No. MO2007A000240, from which this application claims priority, are incorporated herein by reference.

Where technical features mentioned in any claim are followed by reference signs, those reference signs have been included for the sole purpose of increasing the intelligibility of the claims and accordingly such reference signs do not have any limiting effect on the interpretation of each element identified by way of example by such reference signs.

1-19. (canceled)

20. A device for closing lines for administering medical or pharmaceutical fluids from containers and the like, comprising:
a first tubular body and a second tubular body, which are mutually associated at a respective end and are open at the respective opposite ends, said first and second tubular bodies being mutually substantially coaxial and able to move axially by sliding and/or rotating with respect to each other, said first and second tubular bodies having respective free ends associable respectively with the mouth of a container or with a line for administering a fluid and with the mouth of a reservoir for supplying said fluid to said container or to said administration line or to the inlet of a fluid user device,
a valve element for adjusting the flow of said fluid formed inside said first and second tubular bodies, the relative movement of said first and second tubular bodies being adapted to move said valve element from a closed configuration for blocking the flow of said fluid to an open configuration for feeding said fluid from said reservoir to said container, or to said administration line, or from said container, or from said administration line, to said user device, and vice versa,
means for temporary locking in an open and/or closed configuration, which are interposed between said first and second tubular bodies and can be operated by an operator by means of a relative rotation of said tubular bodies, and
further comprising means for limiting the relative rotation between said first and second tubular bodies, said limiting means being monolithically connected to at least one of said first and second tubular bodies.

21. The device according to claim 20, wherein said temporary locking means comprise at least one pin which is associated with at least one of said first and second tubular bodies and is adapted to engage in at least one receptacle associated with the other of said first and second tubular bodies.

22. The device according to claim 21, wherein said locking means comprise at least one pair of said receptacles for temporarily retaining said pin respectively in said open configuration and in said closed configuration.

23. The device according to claim 22, further comprising two of said receptacles for temporarily retaining said pin in said closed configuration, said pin being engageable selectively with one of them depending on the direction of mutual rotation between said first and second tubular bodies that is imparted.

24. The device according to claim 21, wherein said temporary locking means comprise at least one wing which protrudes from said first tubular body substantially parallel to the axis of said first tubular body and faces the outer surface of said second tubular body, said at least one receptacle for said pin being provided on said wing.

25. The device according to claim 24, wherein said locking means comprise at least one pair of said pins, which are aligned along said second tubular body and are spaced by an extent which is equal to the relative sliding stroke between said first and second tubular bodies, said wing being provided with a pair of said receptacles which are adapted to accommodate at least one of said pins.

26. The device according to claim 20, wherein said limiting means are associated externally with at least one of said first and second tubular bodies.

27. The device according to claim 20, wherein said limiting means comprise at least one guide along which said receptacles are formed and in which said pin is inserted slidingly, said pin being associated with at least one of said first and second tubular bodies, said guide being provided on the other of said first and second tubular bodies.

28. The device according to claim 27, wherein said receptacles are formed at the ends of said guide.

29. The device according to claim 27, wherein said guide has at least one connecting portion which is interposed between said receptacles for the continuous transfer of said pin from said open configuration to said closed configuration and vice versa.

30. The device according to claim 29, wherein said connecting portion has a substantially straight longitudinal extension which is parallel to the axis of said second tubular body.

31. The device according to claim 24, wherein said limiting means comprise at least one third tubular body which is rigidly connected, externally and coaxially, to said second tubular body which has at least one notch arranged at said wing.

32. The device according to claim 31, wherein said notch has a longitudinal axis which is substantially parallel to the axis of said third tubular body and has a first lateral abutment wall for said wing in a configuration for the disengagement of said locking means, in which said pins are uncoupled from said respective receptacles, said first wall substantially facing said wing, and a second lateral wall which faces said receptacle and is separated from it in said disengagement configuration, said first and second walls being adapted to interfere with said wing to determine the relative rotation stroke limit, in the two directions of rotation, between said first and second tubular elements.

33. The device according to claim 31, wherein said notch has a width which is at least greater than the width of said wing.

34. The device according to claim 31, wherein a difference in width between said notch and said wing is substantially equal to the mutual rotation stroke between said first and second tubular bodies.

35. The device according to claim 31, wherein said third tubular body is fixed to said second tubular body by means of at least one interlocking coupling which is interposed between said second and third tubular bodies, said interlocking coupling being provided at an internal surface of said third tubular body.

36. The device according to claim 31, wherein said third tubular body is fixed to said second tubular body by means of the interposition of at least one adhesive element.

37. The device according to claim 31, wherein said third tubular body is provided monolithically with said first tubular body.

38. The device according to claim 31, wherein said first tubular body comprises at least one pair of said wings, each of which is provided with a pair of said receptacles, said second tubular body comprising at least two of said pairs of pins which can face said respective receptacles and said third tubular body comprising at least one pair of said notches at said wings.
